(19) United States
(12) Patent Application Publication
(10) Pub. No.: US 2022/0143236 A1
(43) Pub. Date: May 12, 2022

(54) STERILIZATION DEVICE AND STERILIZATION CONTAINER USING SAME

(71) Applicant: IRTRONIX, INC., Torrance, CA (US)

(72) Inventors: Danny OH, Torrance, CA (US); Chien NGUYEN, Santa Ana, CA (US)

(21) Appl. No.: 17/424,444

(22) PCT Filed: Feb. 11, 2020
(86) PCT No.: PCT/KR2020/001928

§ 371 Date: Jan. 19, 2022

Related U.S. Application Data
(60) Provisional application No. 62/804,459, filed on Feb. 12, 2019.

(51) Int. Cl. A61L 2/10; A61L 2/26; C02F 1/32

(52) U.S. Cl. CPC A61L 2/10; A61L 2/26; C02F 1/325; A61L 2202/122; C02F 2307/04; C02F 2201/3222; C02F 2201/3227; C02F 2201/004; C02F 2303/04; A61L 2202/11

(57) ABSTRACT

In a sterilization apparatus and a sterilization container using the same according to an embodiment of the present invention, provided is a sterilization apparatus including: an accommodation unit accommodating an object therein, and having an entrance through which the object enters and exits formed on one side and a connector formed on the other side; a coupling unit supporting the accommodation unit provided in the connector; and a sterilization unit disposed adjacent to the accommodation unit by the coupling unit, and irradiating light to the inside of the accommodation unit through the connector to sterilize the object.

### TECHNICAL FIELD

The present invention relates to a sterilization apparatus and a sterilization container using the same, and more particularly, to a sterilization apparatus in which an LED light source is provided in a lower portion of an accommodation container and a transmission unit provided between the accommodation container and the LED light source, and the LED light source thus sterilizes an object in the accommodation container through the transmission unit, and a sterilization container using the same.

### BACKGROUND ART

In general, as a method for sterilizing water, chemical sterilization by chlorine, heat sterilization, ultraviolet sterilization, ozone sterilization, and the like are known, but the ultraviolet sterilization is widely used due to harmful effects of chemicals and upswell of environmental awareness.

Even if ultraviolet rays are irradiated in water, there is no residue such as the chemicals, and since the virus is inactivated, it is excellent in terms of safety.

In addition, ultraviolet rays are said to destroy virus DNA or RNA, and there is also an advantage in that it does not create resistant bacteria unlike the chemical sterilization.

In recent years, as an interest in personal hygiene has increased, various sterilizer products that sterilize items have been variously released in order to remove germs, bacteria, viruses, etc., from various items that are frequently used and easily contaminated, such as toothbrushes, smartphones, baby bottles, shoes, etc. However, conventional general sterilizers are bulky and can only be used in a fixed position where power can be supplied in a home, and it is difficult to carry.

In particular, commonly used cups or tumblers or infant products used frequently commonly and used by infants and children such as baby bottles, nipples, and the like are simply used only by washing with detergents.

Alternatively, people who react more sensitively possess and use various types of sterilizers for sterilizing treatment by applying a lamp that generates ultraviolet rays for sterilization because sterilization and disinfection effects cannot be obtained only by such washing.

However, when going out, the conventional household sterilizer cannot be carried, and only a sterilized tumbler or baby products are prepared and used at home, but it is difficult to prepare and carry a sufficient amount of feeding bottles or other supplies that are regularly used every fixed times, and as the tumbler as a container containing a liquid is frequently used, the tumbler has an unsanitary problem in that the contaminated container must be continuously reused after one use.

In recent years, as products that apply UV-LED sterilizers to toothbrushes appear, the public's attention is focused.

However, the UV-LED sterilizer mounted on the toothbrush is conceived from the lid of the toothbrush head, so when not in use, a lid is closed and the bristles can be directly irradiated with ultraviolet rays. However, in containers such as tumblers and baby bottles, the liquid inside the container must be washed, so it is very difficult to irradiate infrared rays inside the container.

### DISCLOSURE

### Technical Problem

The present invention was invented to improve the above-described problem, and an object of the present invention relates to a sterilization apparatus for sterilizing a liquid contained in a container such as tumblers or baby bottles, and a sterilization container using the same.

Specifically, an object of the present invention is to provide a sterilization apparatus which is easily carried to enable sterilization externally, and is applicable to various containers to sterilize the inside of the container, and a sterilization container using the same.

The technical objects of the present invention are not limited to the above-mentioned technical objects, and other technical objects not mentioned can be clearly understood by those skilled in the art from the following description.

### Technical Solution

A sterilization container may include: an accommodation unit accommodating an object therein, and having an entrance through which the object enters and exits formed on one side and a connector formed on the other side; a coupling unit supporting the accommodation unit provided in the connector; and a sterilization unit disposed adjacent to the accommodation unit by the coupling unit, and irradiating light to the inside of the accommodation unit through the connector to sterilize the object.

In addition, the sterilization unit may include a light source unit provided in the coupling unit and irradiating LED light to the inside of the accommodation unit, and a transmission unit disposed between the light source unit and the accommodation unit in the coupling unit, and transmitting the LED light.

Further, the light source unit may include one or more LED light sources, and the LED light source may be a UV-C LED light source.

Further, the light source unit may include multiple LED light sources, and the LED light source may include the UV-C LED light source and a Blue LED light source.

Further, the accommodation unit may be provided to open the connector, and the coupling unit may be detachably provided in the accommodation unit.

In addition, the coupling unit may include a first coupling unit closing the connector in the lower portion of the accommodation unit and provided to surround the transmission unit, and a second coupling unit provided in the lower portion of the first coupling unit so that the light source unit is disposed in the lower portion of the transmission unit.

Further, the second coupling unit may be detachably provided in the first coupling unit.

In addition, the coupling unit may have an accommodation groove so as to seat the accommodation unit, and the light source unit may be formed coplanarly with an upper surface of the accommodation groove.

Further, the accommodation unit may be formed to be transparent so as to transmit the LED light of the light source unit.

In addition, the coupling unit may include a power supply unit providing power to the light source unit.

Further, the sterilization container may be provided to be wirelessly chargeable through a separate wireless charging pad.

### Advantageous Effects

According to a sterilization apparatus and a sterilization container using the same of the present invention, the following effects are obtained.

First, an LED is provided to be irradiated toward a container which accommodates a liquid therein to effectively sterilize the liquid in the container.

Second, there is an advantage in that the sterilization apparatus is provided to be coupled to the container to sterilize the liquid even while moving and even while using the container.

Third, there is an advantage in that a power supply unit of the container can be wirelessly charged.

The effects of the present invention are not limited to the aforementioned effect, and other effects, which are not mentioned above, will be apparent to a person having ordinary skill in the art from the description of the claims.

### DESCRIPTION OF DRAWINGS

A detailed description of according to a preferred embodiment of the present application, which is described below and a summary described above will be able to be more well appreciated when reading the detailed description and the summary in association with the accompanying drawings. Preferred embodiments are illustrated in the drawings for the purpose of illustrating the present invention. However, it should be appreciated that the present application is not limited to the illustrated accurate arrangement and means.

FIG. 1 is a diagram illustrating shapes of a sterilization apparatus and a sterilization container using the same, and a wireless charging pad according to an embodiment of the present invention;

FIG. 2 is a plan view illustrating a shape of the inside of an accommodation unit when viewing, from the top, the sterilization apparatus and the sterilization container using the same according to an embodiment of the present invention;

FIG. 3 is a cross-sectional view illustrating the accommodation unit, and a coupling unit and a sterilization unit in the sterilization apparatus and the sterilization container using the same according to an embodiment of the present invention;

FIG. 4 is a diagram illustrating a case where the coupling unit is provided to include a first coupling unit and a second coupling unit in the sterilization apparatus and the sterilization container using the same according to an embodiment of the present invention;

FIG. 5 is a cross-sectional view in which the sterilization apparatus and the sterilization container using the same of FIG. 4 are separated;

FIG. 6 is a cross-sectional view illustrating a case where a sterilization unit is provided in a coupling unit to sterilize the inside of the accommodation unit;

FIG. 7 is a cross-sectional view in which the sterilization apparatus and the sterilization container using the same of FIG. 6 are separated;

FIG. 8 is a cross-sectional view illustrating a case where a first coupling unit is integrally provided in the accommodation unit in the sterilization apparatus and the sterilization container using the same according to an embodiment of the present invention.

FIG. 9 is a cross-sectional view in which the sterilization apparatus and the sterilization container using the same of FIG. 8 are separated;

FIG. 10 is a cross-sectional view illustrating a case where an upper portion of a coupling unit is recessed and an accommodation unit is seated in a sterilization apparatus and a sterilization container using the same according to a modified embodiment of the present invention;

FIG. 11 is a diagram illustrating a case where the accommodation unit is separated from the coupling unit in FIG. 10;

FIG. 12 is a diagram illustrating a case where a transmission unit is integrally formed in a lower portion and the accommodation unit is seated on a coupling unit including a light source unit in the sterilization apparatus and the sterilization container using the same according to the modified embodiment of the present invention;

FIG. 13 is a diagram illustrating a case where the accommodation unit is separated from the coupling unit in FIG. 12; and

FIG. 14 is a conceptual view illustrating a case of sterilizing various accommodation units and cellular phones in a sterilization apparatus and a sterilization container using the same according to various embodiments of the present invention.

### MODE FOR INVENTION

Hereinafter, a preferred embodiment of the present invention, in which a purpose of the present invention can be realized in detail will be described with reference to the accompanying drawings. In describing the embodiment, the same name and the same reference numeral are used with respect to the same component and the resulting additional description will be omitted.

Hereinafter, a preferred embodiment of the present invention, in which a purpose of the present invention can be realized in detail will be described with reference to the accompanying drawings. In describing the embodiment, the same name and the same reference numeral are used with respect to the same component and the resulting additional description will be omitted.

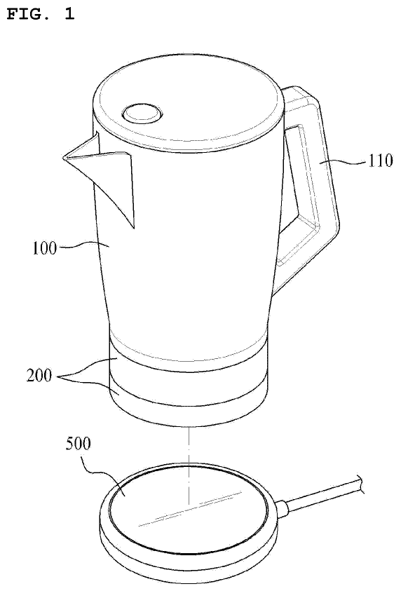

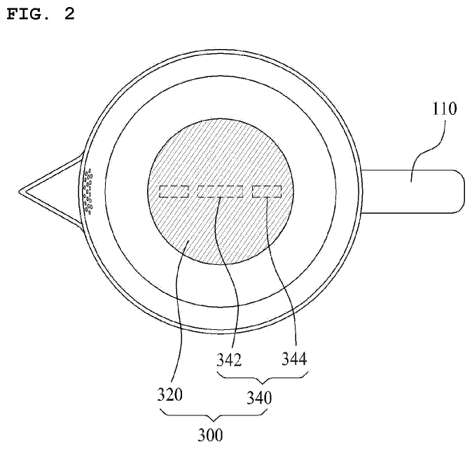

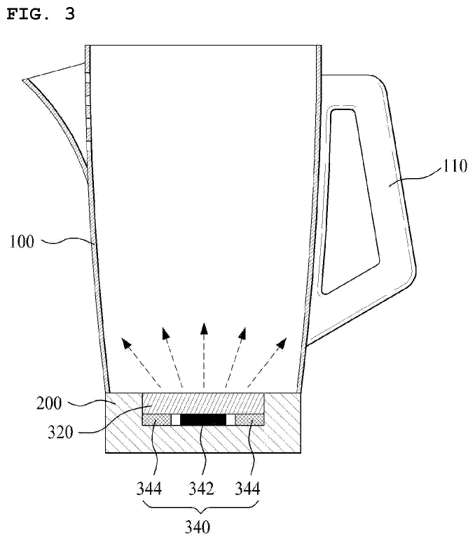

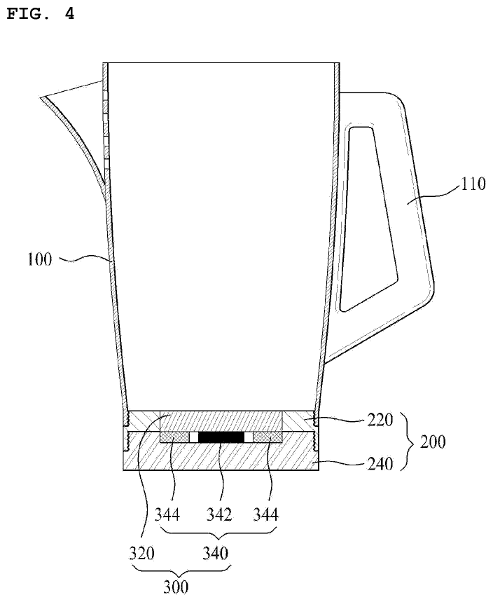

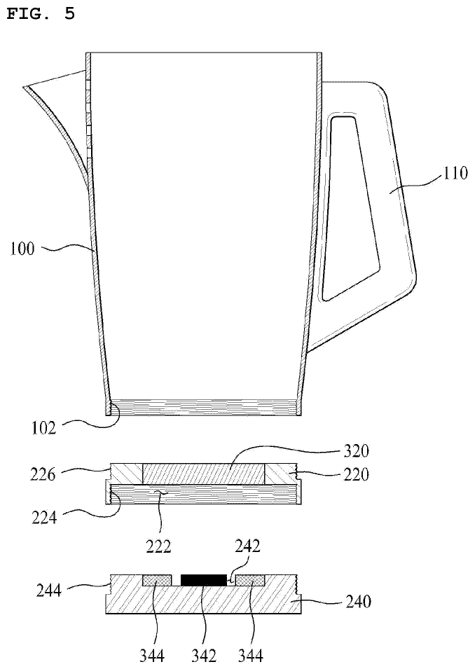

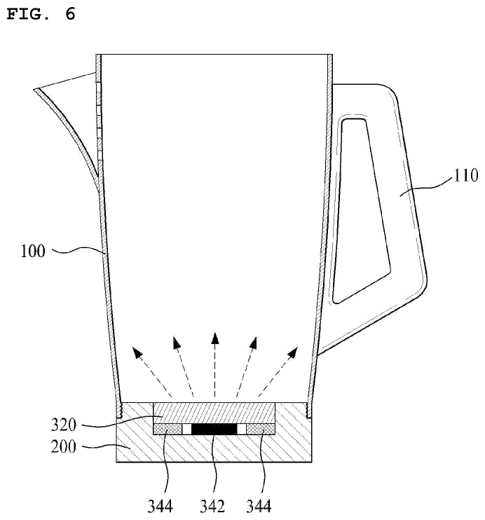

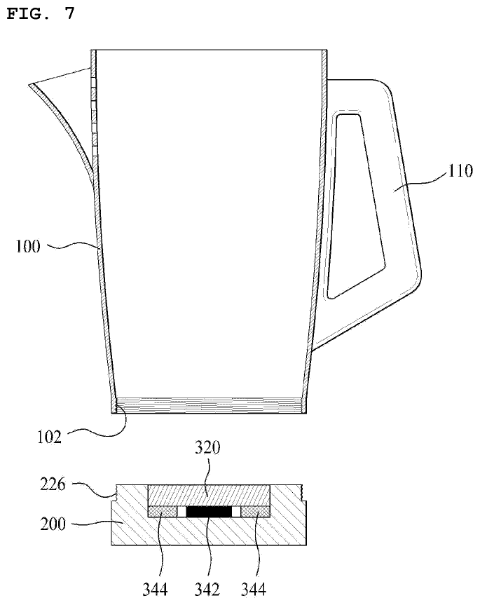

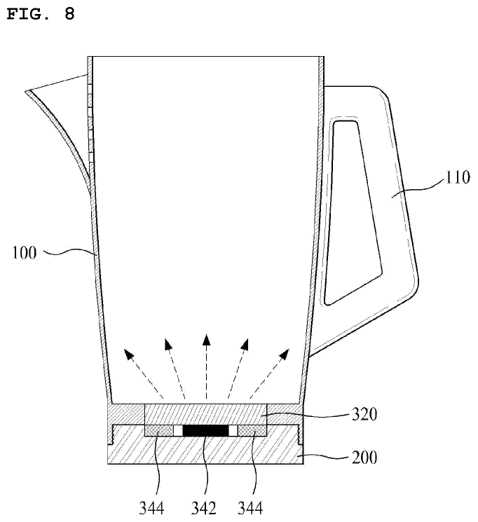

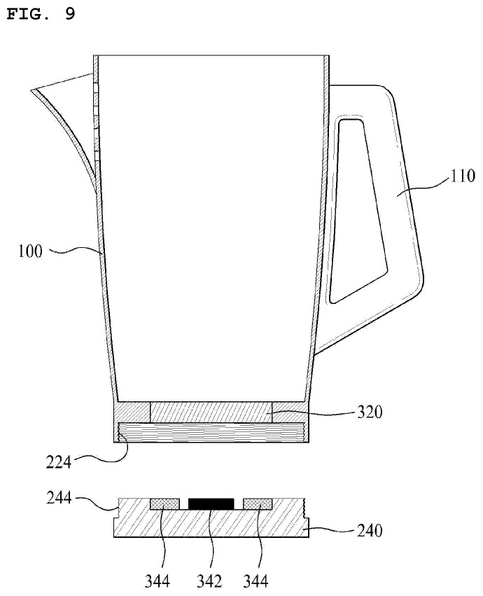

FIG. 1 is a diagram illustrating shapes of a sterilization apparatus and a sterilization container using the same, and a wireless charging pad 500 according to an embodiment of the present invention; FIG. 2 is a plan view illustrating a shape of the inside of an accommodation unit 100 when viewing, from the top, the sterilization apparatus and the sterilization container using the same according to an embodiment of the present invention; FIG. 3 is a cross-sectional view illustrating the accommodation unit 100, and a coupling unit 200 and a sterilization unit 300 in the sterilization apparatus and the sterilization container using the same according to a first embodiment of the present invention; FIG. 4 is a diagram illustrating a case where the coupling unit 200 is provided to include a first coupling unit 220 and a second coupling unit 240 in the sterilization apparatus and the sterilization container using the same according to a second embodiment of the present invention; FIG. 5 is a cross-sectional view in which the sterilization apparatus and the sterilization container using the same of FIG. 4 are separated; FIG. 6 is a cross-sectional view illustrating a case where a sterilization apparatus 300 is provided in a coupling unit 200 to sterilize the inside of an accommodation unit 100 in the sterilization apparatus and the sterilization container using the same according to a first embodiment of the present invention; FIG. 7 is a cross-sectional view in which the sterilization apparatus and the sterilization container using the same of FIG. 6 are separated; FIG. 8 is a cross-sectional view illustrating a case where a first coupling unit 220 is integrally provided in the accommodation unit 100 in the sterilization apparatus and the sterilization container using the same according to a fourth embodiment of the present invention; and FIG. 9 is a cross-sectional view in which the sterilization apparatus and the sterilization container using the same of FIG. 8 are separated.

When a configuration of the present invention is described through FIGS. 1 and 2, the present invention generally includes an accommodation unit 100, a coupling unit 200, and a sterilization unit 300.

The accommodation unit 100 accommodates an object therein, and has an entrance formed on one side, through which the object enters and exits and a connector formed on the other side.

Here, the entrance and the connector may be both sides in which an upper portion and a lower portion are opened in the accommodation unit 100.

That is, in the present invention, the accommodation unit 100 may have a form in which the upper portion and the lower portion are opened.

Further, in the present invention, a container described in Claim 1 may be the same component as the accommodation unit 100 described in Claim 2.

In addition, the coupling unit 200 is provided at a connector at a lower portion of the accommodation unit 100.

The coupling unit 200 supports the accommodation unit 100 and has a form of a frame.

In addition, the sterilization unit 300 is provided in the coupling unit 200 and irradiates light to the inside of the accommodation unit 100 and sterilizes the object accommodated in the accommodation unit 100.

The object described herein may become liquids such as water.

Continuously, the sterilization unit 300 that sterilizes the object in the accommodation unit 100 includes a light source unit 340 provided in the coupling unit 200 and irradiating LED light to the inside of the accommodation unit 100, and a transmission unit 320 disposed between the light source unit 340 and the accommodation unit 100 in the coupling unit 200 and transmitting the LED light.

Here, the transmission unit 320 may adopt quartz glass or a variety of materials which are transparent and through which an LED may be transmitted, and in an embodiment, quartz glass may be used.

The quartz glass is made only of SIO2, and as a result, the amount of metal impurities is extremely small and is at most ppm to several ppm or less. Therefore, there are characteristics that impurities and air bubbles are hardly included, and excellent light transmittance is provided in a wide wavelength range of an ultraviolet to infrared range.

Continuously, the light source unit 340 may be constituted by at least one LED light source, and further, may be provided as a UV-C LED light source which is a first light source 342.

Alternatively, the light source unit 340 may include multiple LED light sources, and at least one may be the UV-C LED light source which the first light source 342 and the other one may be a Blue LED light source which is a second light source 344.

In the embodiment, sterilization may be performed by the first light source 342, and the second light source 344 may be disposed around the first light source 342 for a visual effect.

The first light source 342 as infrared rays is not seen with eyes, so that the second light source 344 electrically connected to the first light source 342 emits light in a visible ray range to check whether the sterilization unit 300 is actuated.

Meanwhile, the coupling unit 200 may accommodate a power supply unit together with the sterilization unit 300.

The power supply unit, which supplies power to the light source unit 340 to allow the light source unit 340 to express the LED light, may be an electrical component such as a circuit board and a capacitor.

Another embodiment of the power supply unit may include a battery to be charged while contacting a separate wireless charging pad 500.

The power supply unit may also be a component into which the battery is inserted.

In a first embodiment of the present invention for carrying out such a component, the accommodation unit 100, the coupling unit 200, and the sterilization unit 300 may be integrally formed as illustrated in FIG. 3.

In FIG. 3, the accommodation unit 100 has a shape of a kettle. A grip portion 110 is formed in a part of a circumference.

According to an embodiment of the present invention, the accommodation unit 100 has the shape of the kettle, but this is just an embodiment of the present invention and all of various containers capable of accommodating the liquid, which include a baby bottle, a tumbler, a water bottle, etc., may become the accommodation unit 100.

Continuously, the coupling unit 200 may be formed on the bottom of the accommodation unit 100 and the transmission unit 320 may be formed in a part of the top of the coupling unit 200.

In addition, the coupling unit 200 may have a form in which the light source unit 340 is disposed in the lower portion of the transmission unit 320.

Here, the accommodation unit 100 may be made of a material which is transparent and through which the LED light may be transmitted and may also be a form which is provided to be opaque and has the transmission unit 320 on a lower surface.

Meanwhile, according to a second embodiment of the present invention, the accommodation unit 100 and the coupling unit 200 may be provided to be coupled to each other.

As illustrated in FIGS. 4 and 5, a first thread 102 may be formed on an inner circumference in the lower portion of the accommodation unit 100 and the coupling unit 200 may be provided to include a first coupling unit 220 and a second coupling unit 240. In addition, the transmission unit 320 and the light source unit 340 may be mounted on the first coupling unit 220 and the second coupling unit 240, respectively.

Specifically, the first coupling unit 220 has the transmission unit 320 provided therein and is provided to surround the transmission unit 320, and a second thread 226 coupled to the first thread 102 may be formed on an outer circumference.

Here, the transmission unit 320 is shown to have a cross section formed by a rectangular parallelepiped, but a shape of the transmission unit 320 may be changed so that the first light source 342 which is passed in the lower portion is evenly diffused to the inside of the container.

For example, when an LED light source passes through the transmission unit 320 in the first light source 342, the transmission unit 320 may be formed in a shape of a concave lens or provided in a modified shape of a Fresnel lens so that the light source is diffused to the entire container.

In addition, the first coupling unit 220 may have a form in which a recessed groove 224 is formed in the lower portion and a third thread 224 is formed on the circumference of the recessed groove 224.

In addition, in the second coupling unit 240, a light emitting groove 242 is formed in the upper portion and the light source unit 340 is seated and while a part is inserted into the recessed groove 224, a fourth thread 244 coupled to the third thread 224 may be formed and the light source unit 340 may be disposed in the upper portion.

Therefore, the second coupling unit 240 is inserted into the recessed groove 224 of the first coupling unit 220, and the light source unit 340 and the transmission unit 320 are disposed to contact each other or to be spaced apart from each other by a predetermined interval, and light irradiated from the light source unit 340 will be irradiated to the inside of the accommodation unit 100 through the transmission unit 320.

Here, the first coupling unit 220 as a portion in which the first thread 102 is formed on the circumference is formed to be stepped to enable a part where the first thread 102 is formed to be inserted into the accommodation unit 100.

In addition, an o-ring made of rubber or synthetic rubber may be provided between the first thread 102 and the second thread 226 in order to prevent moisture from penetrating.

Meanwhile, according to a third embodiment of the present invention, a structure may be provided, in which the sterilization unit 300 is provided in the coupling unit 200 and the coupling unit 200 is coupled to the accommodation unit 100.

As illustrated in FIGS. 6 and 7, the coupling unit 200 is not provided as the first coupling unit 220 and the second coupling unit 240 but integrally provided in the first embodiment.

Therefore, an upper surface of the coupling unit 200 may be formed to be coplanar with one surface of the transmission unit 320, and the light source unit 340 may be provided to be disposed in the lower portion of the transmission unit 320 in the coupling unit 200.

In addition, a structure may be provided, in which the second thread 226 is formed on an upper circumference of the coupling unit 200 and the second thread is coupled to the first thread 102 of the accommodation unit 100.

Meanwhile, according to a fourth embodiment of the present invention, the transmission unit 320 may be integrally formed in the lower portion of the accommodation unit 100.

In addition, a structure may be provided, in which the second coupling unit 240 is coupled to the accommodation unit 100.

As illustrated in FIGS. 8 and 9, the transmission unit 320 is integrally formed on a part of the lower surface of the accommodation unit 100, and as a result, the inside of the accommodation unit 100 and the exterior of the accommodation unit 100 may be projected to each other, and a structure may be provided, in which the recessed groove 224 is formed on the lower surface of the accommodation unit 100, the third thread 224 is formed on the circumference of the recessed groove, the fourth thread 244 formed on the upper circumference of the second coupling unit 240 is coupled to the third thread 224, and the second coupling unit 240 is inserted into the recessed groove 224.

Meanwhile, according to a modified first embodiment, the coupling unit 200 may be provided in a form in which the lower portion of the accommodation unit 100 is seated on the coupling unit 200.

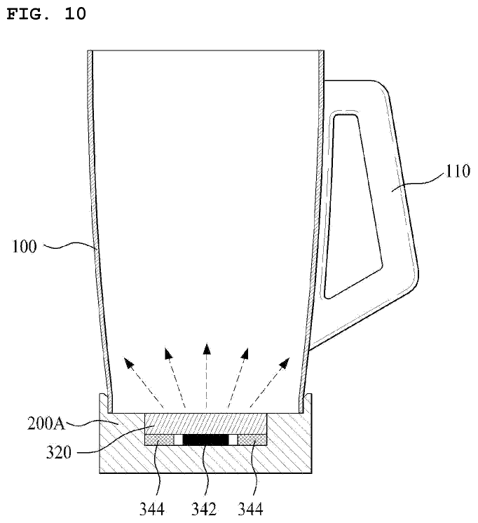

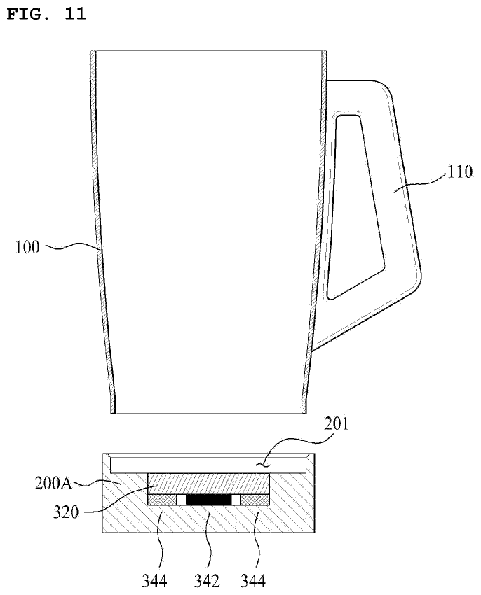

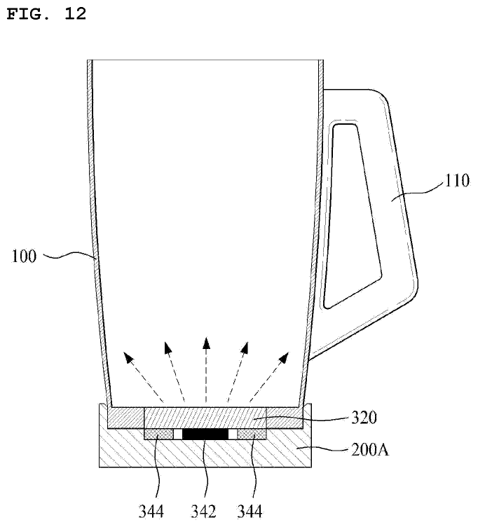

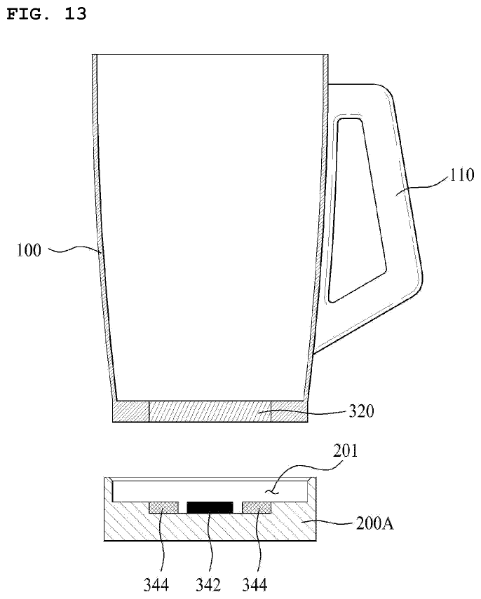

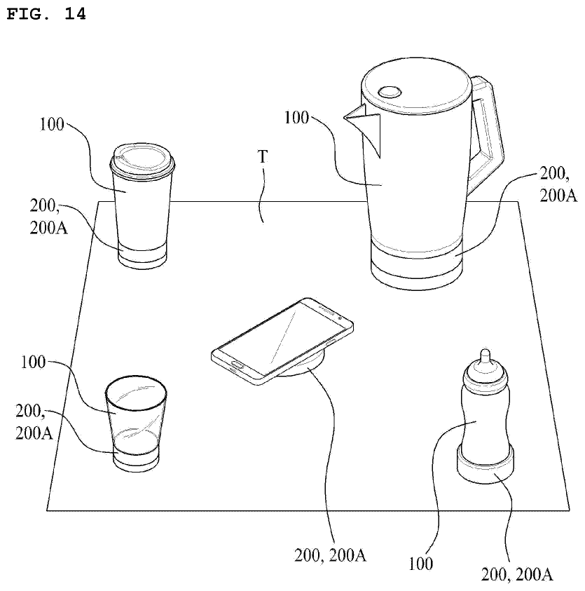

FIG. 10 is a cross-sectional view illustrating a case where an upper portion of a coupling unit 200 is recessed and an accommodation unit 100 is seated in a sterilization apparatus and a sterilization container using the same according to a first modified embodiment of the present invention; FIG. 11 is a diagram illustrating a case where the accommodation unit 100 is separated from the coupling unit 200 in FIG. 10; FIG. 12 is a diagram illustrating a case where a transmission unit 320 is integrally formed in a lower portion and the accommodation unit 100 is seated on a coupling unit 200 including a light source unit 340 in the sterilization apparatus and the sterilization container using the same according to the modified second embodiment of the present invention; FIG. 13 is a diagram illustrating a case where the accommodation unit 100 is separated from the coupling unit 200 in FIG. 12; and FIG. 14 is a conceptual view illustrating a case of sterilizing various accommodation units 100 and cellular phones in a sterilization apparatus and a sterilization container using the same according to various embodiments of the present invention.

First, referring to FIGS. 10 and 11, a seating groove 201 may be formed in the upper portion of a coupling unit 200A and the accommodation unit 100 may be seated, and the light source unit 340 and the transmission unit 320 may be provided in the coupling unit 200A.

Specifically, the light source unit 340 may be disposed in the coupling unit 200A and the transmission unit 320 may be disposed in the upper portion of the light source unit 340, and the transmission unit 320 may contact the accommodation unit 100. In addition, here, the accommodation unit 100 may be provided as a transparent material, and may be a material through which the LED light source of the light source unit 340 is transmitted.

In addition, the coupling unit 200A may have the power supply unit provided therein so as to supply the power to the light source unit 340 and may be wirelessly charged through a separate wireless charging pad 500.

Further, the power supply unit includes the battery to have a charging form and may have a form in which the battery is inserted.

Therefore, when a user seats the accommodation unit 100 on the seating groove of the coupling unit 200A according to the modified first embodiment of the present invention and provides the power, it may be convenient for the user to sterilize the object in the accommodation unit 100.

Meanwhile, the transmission unit 320 may be integrally provided in an accommodation unit 100 according to a modified second embodiment, the coupling unit 200A has the recessed groove to seat the accommodation unit 100, and the light source unit 340 may be provided in the coupling unit 200A.

Referring to FIGS. 12 and 13, a seating groove 201 may be formed and the accommodation unit 100 may be seated on the upper portion of the coupling unit 200A.

In this case, the light source unit 340 and the light source unit 340 which is coplanar with the lower surface of the accommodation unit 100 may be adjacent to each other, and the LED light irradiated from the light source unit 340 may be irradiated to the inside of the accommodation unit 100 through the transmission unit 320.

According to the modified first embodiment and the modified second embodiment, a structure may be provided, in which the coupling unit 200A and the accommodation unit 100 are coupled to each other.

Specifically, a thread is formed on a lower outer circumference of the accommodation unit 100 and a thread is formed on the circumference of the recessed groove, and the accommodation unit and the coupling unit are coupled to each other.

Further, in the embodiment, a form is provided, in which the accommodation unit 100 and the coupling unit 200A are coupled to each other by the thread, but this is just one embodiment, and is changeable to force fitting, a coupling form of a protrusion and a groove, etc., and for a coupling scheme, the scope of the present invention is not limited.

Next, FIG. 14 is a diagram illustrating various embodiments of the sterilization apparatus and the sterilization container using the same according to the present invention.

On a table T, various forms of sterilization apparatuses and a sterilization container using the same are disposed.

In the first to fourth embodiments of the present invention, the accommodation unit 100 and the coupling units 200 and 200A are coupled, and as a result, the sterilization apparatus may be provided to be mounted on an accommodation container such as a tumbler, a mug, a glass cup, a bay bottle, or a glass kettle.

In such a form, the battery may be provided or the sterilization apparatus may be chargeable by the wireless charging pad 500.

In addition, according to the modified first and second embodiments of the present invention, when the coupling units 200 and 200A having the recessed groove 224 are provided in the room and the accommodation unit 100 is seated on the coupling units 200 and 200A, the object in the accommodation unit 100 may be sterilized.

A preferred embodiment of the present invention has been described as above and a fact that the present invention can be materialized in other specific forms without departing from the gist or scope in addition to the above described embodiment is apparent to those skilled in the art. Therefore, the aforementioned embodiment is not limited but should be considered to be illustrative, and as a result, the present invention is not limited to the above description and may be modified within the scope of the appended claims and a range equivalent thereto.

1. A sterilization apparatus for sterilizing an object accommodated in a container, comprising:
a coupling unit provided to be in close contact with the container; and
a sterilization unit formed in the container and emitting light toward the object, and provided to transmit the light to the inside of the container.

2. A sterilization container comprising:
an accommodation unit accommodating an object therein, and having an entrance through which the object enters and exits formed on one side and a connector formed on the other side;
a coupling unit supporting the accommodation unit provided in the connector; and
a sterilization unit disposed adjacent to the accommodation unit by the coupling unit, and irradiating light to the inside of the accommodation unit through the connector to sterilize the object.

3. The sterilization container of claim 2, wherein the sterilization unit includes
a light source unit provided in the coupling unit and irradiating LED light to the inside of the accommodation unit, and
a transmission unit disposed between the light source unit and the accommodation unit in the coupling unit, and transmitting the LED light.

4. The sterilization container of claim 3, wherein the light source unit includes at least one LED light source, and
the LED light source is a UV-C LED light source.

5. The sterilization container of claim 3, wherein the light source unit includes multiple LED light sources, and
the LED light source includes the UV-C LED light source and a Blue LED light source.

6. The sterilization container of claim 3, wherein the accommodation unit is provided to open the connector, and
the coupling unit is detachably provided in the accommodation unit.

7. The sterilization container of claim 6, wherein the coupling unit includes
a first coupling unit closing the connector in the lower portion of the accommodation unit and provided to surround the transmission unit, and
a second coupling unit provided in the lower portion of the first coupling unit so that the light source unit is disposed in the lower portion of the transmission unit.

8. The sterilization container of claim 7, wherein the second coupling unit is detachably provided in the first coupling unit.

9. The sterilization container of claim 3, wherein the coupling unit has an accommodation groove so as to seat the accommodation unit, and
the light source unit is formed coplanarly with an upper surface of the accommodation groove.

10. The sterilization container of claim 9, wherein the accommodation unit is formed to be transparent so as to transmit the LED light of the light source unit.

11. The sterilization container of claim 2, wherein the coupling unit includes a power supply unit providing power to the light source unit.

12. The sterilization container of claim 11, wherein the sterilization container is provided to be wirelessly chargeable through a separate wireless charging pad.
